(19) United States
(12) Patent Application Publication
(10) Pub. No.: US 2023/0017516 A1
(43) Pub. Date: Jan. 19, 2023

(54) Ventilator

(71) Applicant: Ecological Services International, Inc., Los Fresnos, TX (US)

(72) Inventor: Paul Seales, Port Isabel, TX (US)

(21) Appl. No.: 17/863,704

(22) Filed: Jul. 13, 2022

Related U.S. Application Data
(60) Provisional application No. 63/223,313, filed on Jul. 19, 2021.

(51) Int. Cl. A61M 16/08; A61M 16/00

(52) U.S. Cl. CPC A61M 16/0883; A61M 16/0051; A61M 16/0075

(57) ABSTRACT

A ventilator that moves breathable air into and out of the lungs of a patient. The ventilator includes an inspiratory circuit with an inlet, a bellows, and an outlet port for moving air into the lungs of the patient. An expiratory circuit includes an inlet port and a discharge port for moving the air out of the lungs. The inspiratory circuit is adjustable to control a number of breathes per time period and a volume of the breaths.

### RELATED APPLICATIONS

This application claims the benefit of U.S. Provisional Application No. 63/223,313 filed Jul. 19, 2021, the disclosure of which is incorporated herein by reference in its entirety.

### BACKGROUND

Ventilators are machines that move air into and out of the lungs of a patient. Ventilators are used on patients with breathing issues caused from a variety of health conditions, such as but not limited to covid, acute respiratory distress syndrome (ARDS), chronic obstructive pulmonary disease (COPD), asthma, and pneumonia.

Many ventilators are relatively complicated devices with complex mechanical, electrical, and software components. This complexity can make it difficult to training persons who will be using the ventilator, especially when the persons do not have an extensive medical background.

The complexity of the ventilators can also make them difficult to manufacturer. More precise engineering and assembly is necessary to ensure proper functioning of the ventilators. Further, the materials needed to produce the complex components may be difficult to obtain and/or expensive. Supply chain issues of these components can greatly increase the time necessary to manufacturer the ventilators.

### SUMMARY

One aspect is directed to a ventilator comprising an inspiratory circuit comprising an inlet, a bellows, and an outlet port. An expiratory circuit comprises an inlet port and a discharge port. The inspiratory circuit is adjustable to control a number of breathes per time period and a volume of the breaths.

In another aspect, a control module is configured to control the number of breathes per time period that are output from the inspiratory circuit through the outlet port and the volume of the breaths.

In another aspect, the control module prevents the inspiratory circuit from moving air out of the outlet port concurrently with the expiratory circuit moving air from the patient into the inlet port.

In another aspect, the expiratory circuit comprises a disinfectant chamber positioned downstream from the inlet port and upstream from the discharge port with the disinfectant chamber comprising a light source that emits UV light.

In another aspect, a rotating cam and a cam follower that rides along the cam is operatively connected to the bellows to move the bellows between a contracted configuration and an expanded configuration to move the air through the inspiratory circuit.

In another aspect, an arm is adjustable to control a size of the bellows in an expanded position to control the volume of the breaths.

In another aspect, an alarm is configured to emit an alarm when the bellows falls below a predetermined size.

In another aspect, a housing extends around and protects the inspiratory and expiratory circuits with the housing comprising a front face with the outlet port of the inspiratory circuit and the inlet port of the expiratory circuit each positioned along the front face.

One aspect is directed to a ventilator comprising an inspiratory circuit comprising an inspiratory inlet port to receive air from the environment, bellows positioned downstream from the inspiratory inlet port and movable between an expanded configuration and a contracted configuration, and an outlet port positioned downstream from the bellows to deliver the air to the patient. An expiratory circuit comprises an expiratory inlet port to receive the air that is exhaled from the patient, and a discharge port to discharge the exhaled air. A control module comprises a cam to drive the bellows to adjust a number of breathes supplied to the patient and a lever that contacts against the bellows to control a size of the bellows in the expanded configuration to control an amount of air in each of the breaths.

In another aspect, an alarm sounds an audible signal when the bellows contracts to a size that is smaller than a predetermined amount.

In another aspect, the alarm comprises an arm that is pivotally mounted and contacts against the bellows.

In another aspect, the expiratory circuit comprises a disinfectant chamber positioned downstream from the expiratory inlet port and upstream from the discharge port with the disinfectant chamber comprising a light source that emits UV light.

In another aspect, a cam follower rides along the cam and is operatively connected to the bellows to move the bellows between the contracted configuration and the expanded configuration to move the air through the inspiratory circuit.

In another aspect, an arm is positioned adjacent to the bellows to contact against the bellows to control a size of the bellows in the expanded configuration to control the volume of the breaths.

In another aspect, a housing extends around and protects the inspiratory and expiratory circuits with the housing comprising a front face with the outlet port of the inspiratory circuit and the expiratory inlet port of the expiratory circuit each positioned along the front face.

One aspect is directed to a method of supplying air to a patient. The method comprises: expanding a bellows and in response drawing air into an inlet of the ventilator; contracting the bellows and in response moving air that has been drawn into the ventilator out through an outlet port and to the patient; receiving air that is exhaled from the patient through an inlet port; controlling a speed at which the bellows expands and contracts and controlling a number of breaths of the air that are supplied to the patient through the outlet port; and controlling a size of the bellows in the expanded configuration and controlling an amount of the air that is supplied to the patient in each of the breaths.

In another aspect, the method comprises supplying the air to the patient and simultaneously receiving air that is exhaled from the patient through an inlet port.

In another aspect, the method comprises adjusting an arm that is positioned adjacent to the bellows and controlling the size of the bellows in the expanded configuration.

One aspect is directed to a ventilator comprising an inspiratory circuit comprising an inlet, a bellows, and an outlet port. An expiratory circuit comprises an inlet port, and a discharge port. A control module is configured to control a number of breathes per time period that are output from the inspiratory circuit through the outlet port and a volume of the breaths.

In another aspect, the expiratory circuit comprises a disinfectant chamber that emits UV light.

In another aspect, the control module comprises a rotating cam, and a cam follower that rides along the cam is operatively connected to the bellows to move the bellows between a contracted position and an expanded position to move air through the inspiratory circuit.

In another aspect, the control module prevents the inspiratory circuit from moving air out of the outlet port concurrently with the expiratory circuit moving air from the patient into the inlet port.

In another aspect, the control module comprises an arm that is adjustable to control a size of the bellows in an expanded position to control the volume of the breaths.

In another aspect, conduits extend along the inspiratory and expiratory circuits.

In another aspect, an alarm monitors a size of the bellows.

In another aspect, the alarm sounds an audible signal when the bellows falls below a predetermined size.

In another aspect, a housing extends around and protects the inspiratory and expiratory circuits.

One aspect is directed to a ventilator comprising: an inspiratory circuit comprising an inlet to receive air from the environment; bellows positioned downstream from the inlet and movable between an expanded configuration and a contracted configuration; and an outlet port positioned downstream from the bellows to deliver the air to the patient. An expiratory circuit comprises an inlet port to receive the air that is exhaled from the patient, and a discharge port to discharge the exhaled air. A control module comprises a cam to drive the bellows to adjust a number of breathes supplied to the patient, and a lever that contacts against the bellows to control a size of the bellows in the expanded configuration to control an amount of air in each of the breaths.

In another aspect, an alarm sounds an audible signal when the bellows contracts to a size that is smaller than a predetermined amount.

In another aspect, the alarm comprises an arm that is pivotally mounted and contacts against the bellows.

One aspect is directed to a method of supplying air to a patient comprising: adjusting a first input device and controlling a frequency of a bellows moving through a breath cycle that includes expanded and contracted configurations; and adjusting a second input device and controlling a size of the bellows in the expanded configuration and controlling an amount of air that is supplied to the patient.

One aspect is directed to a method of supplying air to a patient comprising: expanding a bellows and in response drawing air into an inlet of the ventilator; contracting the bellows and in response moving the air that has been drawn into the ventilator out through an outlet port and to the patient; simultaneously receiving air that is exhaled from the patient through an inlet port; controlling a speed at which the bellows expands and contracts and controlling a number of breaths of the air that are supplied to the patient through the outlet port; and controlling a size of the bellows in the expanded configuration and controlling an amount of the air that is supplied to the patient in each of the breaths.

One aspect is directed to a method of supplying air to a patient comprising: adjusting a speed at which a bellows moves between an expanded configuration and a contracted configuration; adjusting a size of the bellows in the expanded configuration; moving the bellows from the contracted configuration to the expanded configuration and drawing air into an inlet of the ventilator; contracting the bellows and moving the air that has been drawn into the ventilator out through an outlet port and to the patient; and simultaneously receiving air that is exhaled from the patient through an inlet port.

### BRIEF DESCRIPTION OF THE DRAWINGS

FIG. 1 is a schematic diagram of a ventilator.

FIG. 2 is a schematic diagram of a ventilator.

FIG. 3 is a schematic diagram of hoses attached to a ventilator and also attached to a mask.

FIG. 4 is a front perspective view of a ventilator.

FIG. 5 is a perspective view of internal components of a ventilator.

FIG. 6 is a perspective view of internal components of a ventilator.

FIG. 7 is a flowchart diagram of a method of supplying air to a patient.

FIG. 8 is a flowchart diagram of a method of supplying air to a patient.

### DETAILED DESCRIPTION

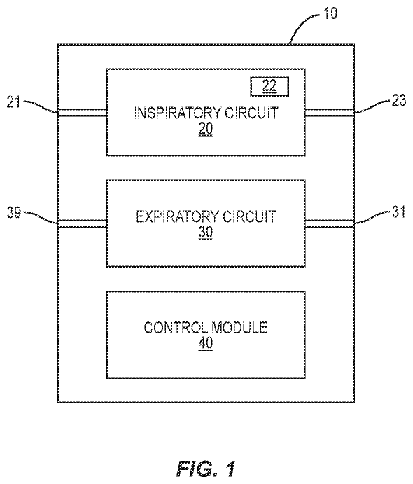

The present application is directed to ventilator that supports or takes over the breathing process of a patient. The ventilator is configured to pump air into the lungs of the patient. The ventilator also receives exhaled air from the patient. FIG. 1 schematically illustrates a ventilator 10 that includes an inspiratory circuit 20 that has an inlet 21, a bellows 22, and an outlet port 23. During use, the bellows 22 moves through a breathing cycle with each breath moving between expanded and contracted configurations to pull air into the inlet 21 and move the air out through the outlet port 23 to the patient. The ventilator 10 also includes an expiratory circuit 30 that includes an inlet port 31 to receive air that is exhaled from the patient and a discharge port 39 to expel the air out of the ventilator 10. A control module 40 is configured to control a number of breaths the bellows 22 moves through within a given time period (e.g., breaths per minute). The control module 40 also controls a size of the bellows 22 in the expanded configuration and thus a volume of air that is provided to the patient in each breath.

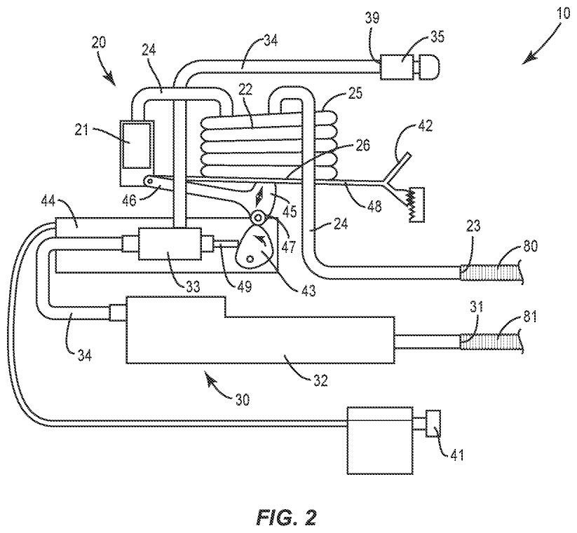

FIG. 2 schematically illustrates the ventilator 10 that includes the inspiratory circuit 20 and the expiratory circuit 30. The inspiratory circuit 20 includes the inlet 21 that receives air from the environment. An air filter can be positioned at the inlet 21 to filter the incoming air. The bellows 22 is positioned downstream from the inlet 21 and receives the air through a conduit 24. The bellows 22 is configured to expand and contract to control the air moving through the inspiratory circuit 20. A conduit 24 from the bellows 22 leads to the outlet port 23 to supply the air to the patient. The outlet port 23 is configured to receive a hose to supply the air to the patient.

The bellows 22 includes an expandable body with an enclosed interior space that receives the air. In one example, the body has an accordion configuration with a number of pleats that move between the expanded and contracted configurations. In another example, the bellows 22 is an expandable material, such as rubber or silicone, that can stretch and rebound depending upon the amount of air within the interior space. The size of the bellows 22 changes depending upon the amount of air within the interior space. The bellows 22 includes a larger size in the expanded configuration and a smaller size in the contracted configuration. In one example, the bellows 22 includes a top side 25 and a bottom side 26 that both move when the bellows 22 expands and contracts.

Each expansion and contraction of the bellows 22 corresponds to one breathing cycle of the patient. The amount of air that is pulled into the bellows 22 during expansion and is forced out of the bellows 22 during contraction corresponds to the amount of air that is supplied to the patient during a breathing cycle.

The expiratory circuit 30 includes the inlet port 31 configured to mount a hose to receive the air that is exhaled from the patient. A conduit 34 formed by one or more sections leads to a discharge port 39 where the exhaled air exits the ventilator 10 and is returned to the environment.

In one example, the expiratory circuit 30 includes a disinfecting chamber 32 to disinfect the exhaled air from the patient prior to the exhaled air being released into the environment. In the example of FIG. 2, the disinfectant chamber 32 is positioned immediately downstream from the inlet port 31. In one example, the disinfectant chamber 32 includes a UV light source that emits light while the air passes to kill germs such as various bacteria and viruses.

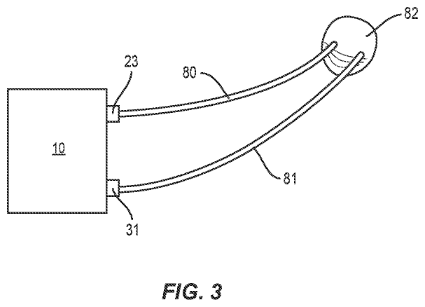

In one example, the expiratory circuit 30 includes a control valve 33 that controls the timing of the expiratory circuit 30 relative to the inspiratory circuit 20. The control valve 33 blocks the expiratory circuit 30 during a positive cycle of the inspiratory cycle of the patient. This ensures that air that is delivered by the ventilator 10 is received into the patient and is not directly returned to the ventilator 10. As illustrated in FIG. 3, a first hose 80 is mounted to the outlet port 23 and leads to a mask 82. A second hose 80 leads from the mask 82 to the inlet port 31. During proper usage, air from the ventilator 10 moves through the hose 80, into the mask 82 where it is inhaled by the patient. Air that is exhaled by the patient moves through the hose 81 and back into the ventilator 10. If the ventilator 10 was to operate with both the inspiratory and expiratory circuits 20, 30 being open, the air from the hose 80 could move directly into the hose 81 without being inhaled by the patient. Thus, the control valve 33 blocks the expiratory circuit 30 when air is being expelled by the inspiratory circuit 20 to prevent this occurrence.

Returning to FIG. 2, the discharge port 39 is positioned downstream from the control valve 33. A peep valve 35 is positioned at the discharge port 39 to maintain positive pressure in the expiratory circuit 30 against which the patient exhales.

The control module 40 controls the timing aspects of the ventilator 10 and the amount of air that is supplied by the ventilator 10 to the patient. The control module 40 includes the first input device 41 to adjust the number of breathes per minute that are supplied through the outlet port 23 to the patient. The second input device 42 controls the volume of air in each breath supplied through the outlet port 23 to the patient.

The first input device 41 controls a motor 44 located in the ventilator 10 that rotates a cam 43. The cam 43 includes a first lobe with a first shape and a second lobe with a second shape. The ventilator 10 also includes a cam follower 45 that rides along the surface of the first lobe of the cam 43. The cam follower 45 is further connected to the bellows 22.

During use, the operator adjusts the first input device 41 to control the number of breaths that are output per minute to the patient. The first input device 41 causes the motor 44 to adjust the rotational speed of the cam 43. During each rotation of the cam 43, the cam follower 45 rides along the cam surface of the first lobe and causes the bellows 22 to expand and contract. During expansion, the bellows 22 causes air to be pulled into the inlet 21 and moved along the upstream conduit 24 towards the bellows 22. During contraction, the air in the bellows 22 is forced through the downstream conduit 24 and out through the outlet port 23 to the patient.

In one example as illustrated in FIG. 2, the cam follower 45 includes a pivoting arm 46. The cam follower 45 further includes a contact member 47 that rides along the surface of the first lobe of the cam 43 during the rotation of the cam 43.

The second input device 42 controls the volume of air that is supplied in each breath. The second input device 42 includes an arm 48 that is mounted in the ventilator 10 and movable relative to the bellows 22. The arm 48 is moved to a position relative to the bellows 22 to limit the expansion of the bellows 22 in the expanded configuration. At one setting, the arm 48 is positioned at a first position relative to the bellows 22 to allow the bellows to expand to a first size to hold a first volume of air. At a second setting, the arm 48 is positioned at a second position that restricts the expansion of the bellows 22 a greater amount for the bellows 22 to only hold a smaller second volume of air. Thus, the positioning of the arm 48 blocks the travel of the bellows 22 beyond a predetermined amount thus controlling the amount of air that the bellows 22 holds and thus expels.

The second input device 42 can control the movement of the arm 48 in various manners. In one example as illustrated in FIG. 2, the arm 48 includes teeth that engage with a track of teeth to control the position of the arm 48. In another example, the arm 48 includes a threaded member that can be rotated to move the angular position of the arm 48 relative to the bellows 22. In another example, a track of detents are aligned in a row and the arm 48 can be lifted and moved to the desired detent to control its position relative to the bellows.

With the first and second input devices 41, 42 properly adjusted, the ventilator 10 operates by the motor 44 rotating the cam 43. This rotation causes the cam follower 45 to move along the surface of the first lobe of the cam 43 and cause the bellows 22 to expand to pull in air from the environment and contract to force the air out through the outlet port 23 to the patient.

Simultaneously, exhaled air from the patient is received through the inlet port 31. The air is moved into the disinfecting chamber 32 where germs, bacteria, and other potentially harmful organism are killed or otherwise disabled. The disinfected air is then moved through the conduit 34, through the valve 33 and through the downstream conduit 34 to the discharge port 39. In one example, the discharge port 39 includes a peep valve 35 that is a spring-loaded valve that maintains pressure on the exhaled air. The peep valve 35 maintains pressure on the lungs of the patient such that at the end of the expiratory phase of the breathing cycle, the pressure in the lungs in slightly above atmospheric pressure in the environment.

As explained above, the control valve 33 blocks the movement of the air through the conduit 34 during a positive inspiratory phase of the breath cycle. A connector 49 extends between the control valve 33 and a second lobe of the cam 43. When the cam 43 is in the rotational position in which air is being forced to the patient (i.e., when the bellows 22 are being contracted), the connector 49 that rides along the second lobe and closes the control valve 33. Once the positive inspiratory phase of the breathing cycle is complete which corresponds to a rotational position of the cam 43 and the start of the expansion of the bellows 22, the connector 49 is positioned on a section of the second lobe of the cam that causes the control valve 33 to open to allow the air to move along the expiratory circuit 30.

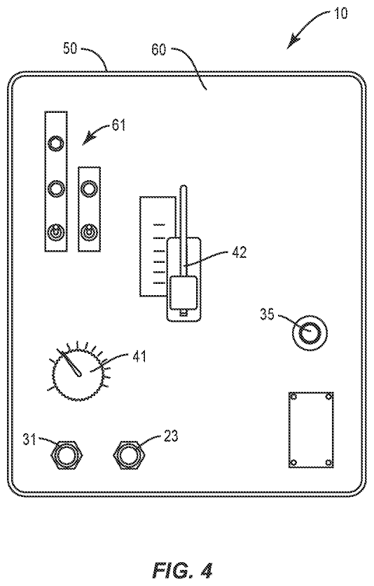

In one example as illustrated in FIG. 4, the ventilator 10 is contained within a housing 50. The housing 50 extends around one or more sides to contain and protect the internal components. A control panel 60 on the housing 50 provides for medical personnel to control the operation of the ventilator 10. In one example as illustrated in FIG. 4, the control panel 60 extends across one face of the housing 50. The control panel 60 includes the first input device 41, such as a rotatable knob, for selectively adjusting the number of breathes per minute in which air from the ventilator 10 is supplied through the outlet port 23 to the patient. The second input device 42 is also positioned on the control panel 60 to selectively adjust the volume of air that is supplied to the patient during each breath. The outlet port 23 and inlet port 31 are positioned on the control panel 60. Each port 23, 31 includes a fitting to receive a conduit, such as a flexible hose 80, 81, to move the air to and from the patient. The peep valve 35 can also be positioned on the control panel 60. Various other input devices 61 on the control panel 60 control various aspects of the ventilator 10, including but not limited to powering on and off, resetting the ventilator, and various alarms.

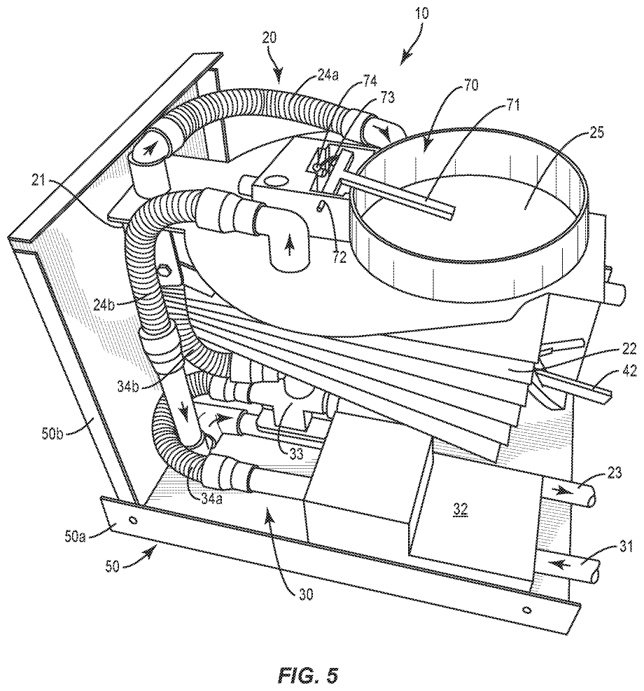

FIG. 5 illustrates a portion of the ventilator with sections of the housing 50 removed with the internal components of the ventilator 10 visible. A bottom wall 50a and a back wall 50b of the housing 50 remain. The inspiratory circuit 20 includes the air inlet 21 positioned at the back wall 50b. A first conduit 24a leads from the air inlet 21 to the bellows 22, and a second conduit 24b leads from the bellows 22 to the outlet port 23 at a front of the housing 50. In this example, both conduits 24a, 24b are positioned at a top of the bellows 22. This positioning provides for drawing air into the bellows 22 through the air inlet 21 and conduit 24a and forcing the air out through the conduit 24b and outlet port 23.

The expiratory circuit 30 also extends through the housing 50 and includes the inlet port 31 at the front of the housing 50. The exhaled air moves from the inlet port 31 into the disinfectant chamber 32. The air then moves through a conduit 34a, through the control valve 33, and through a conduit 34b to the peep valve 35 (not illustrated in FIG. 5). In this example, the peep valve 35 is positioned at the front of the housing 50 (see FIG. 4).

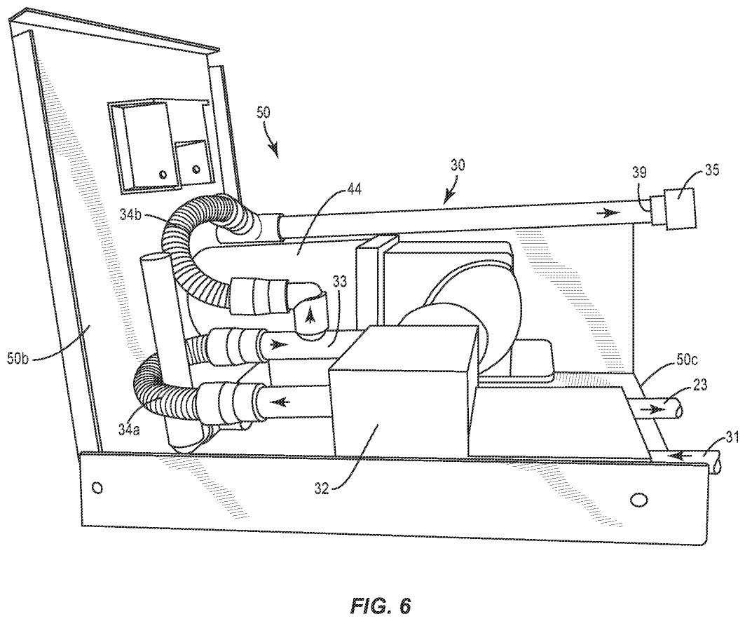

FIG. 6 illustrates another view of the expiratory circuit 30 positioned in the housing 50. In this illustration, other components of the ventilator 10 have been removed for clarity. The expiratory circuit 30 includes the inlet port 31, disinfectant chamber 32, conduits 34a, 34b, control valve 33, and peep valve 35.

In one example as illustrated in FIG. 5, the ventilator 10 includes an alarm 70 to ensure the air pressure in the inspiratory circuit 20 remains above an ambient level. The alarm 70 includes an arm 71 that is mounted at a pivot 72. The arm 71 rests against the top side 25 of the bellows 22. A circuit 73 that is powered by a separate power source 74, such as a battery, monitors the angular position of the arm 71 as it moves about the pivot 72. When the ventilator 10 is functioning properly, air pressure in the bellows 22 causes the bellows 22 to remain inflated and the arm 71 remains within a predetermined angular range. In the event that the air pressure in the inspiratory circuit 20 falls below a predetermined amount, the bellows 22 reduces in size causing the top side 25 to fall and the arm 71 to pivot beyond the predetermined angular range. The circuit 73 monitors the angular position and is configured to cause an alarm to sound through one or more speakers in the event the angular position falls outside of the predetermined range.

In one specific example, the circuit 73 monitors the angular position of the arm 71. When the air pressure in the inspiratory circuit 20 goes to zero or ambient pressure, the bellows 22 reduce in size causing the arm 71 to pivot out of the predetermined range. The alarm will be sounded within a predetermined time period (e.g., 15 seconds) of the air pressure reaching ambient.

The alarm 70 is powered by a separate power source 74 than the motor 44. In one example, the power source 74 is a battery, such as a 11.8 volt battery. In the event of a power failure that causes the motor 44 to stop operating, the alarm 70 is still functional and will sound an audible signal to inform medical personnel in the area about the issue. In one example, the battery 74 is accessible in the housing 50 and can be periodically replaced as necessary.

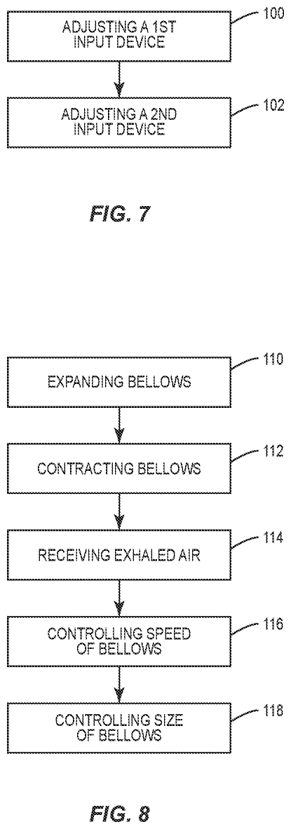

FIG. 7 illustrates a method of supplying air to a patient. The method includes adjusting a first input device 41 and controlling a frequency of a bellows 22 moving through a breath cycle that includes expanded and contracted configurations (block 100). The method also includes adjusting a second input device 42 and controlling a size of the bellows 22 in the expanded configuration and controlling an amount of air that is supplied to the patient (block 102).

FIG. 8 illustrates another method of supplying air to a patient. The method includes expanding a bellows 22 and in response drawing air into an inlet 21 of the ventilator 10 (block 110). The method includes contracting the bellows 22 and in response moving the air that has been drawn into the ventilator 10 out through an outlet port 21 and to the patient (block 112). The method includes receiving air that is exhaled from the patient through an inlet port 31 (block 114). The method also includes controlling a speed at which the bellows 22 expands and contracts and controlling a number of breaths of the air that are supplied to the patient through the outlet port 23 (block 116). Further, the method controls a size of the bellows 22 in the expanded configuration and to control an amount of the air that is supplied to the patient in each of the breaths (block 118).

In one example, one or both of the inspiratory circuit 20 and the expiratory circuit 30 are configured to ensure that air flows in just one direction. This can include valves or other like mechanisms that control the direction of air flow.

The ventilator 10 includes numerous advantages over other similar devices. In one example, the ventilator 10 includes no computer or digital controls. In one example, the ventilator 10 has years of shelf life and is not affected by weather or temperature. In one example, the ventilator 10 is straight-forward to operate and requires a very short operator training period. In one example, the ventilator 10 includes intuitive controls. In one example, the ventilator 10 includes a motor 44 that is powered by a 110 volt power input. In one example, the ventilator 10 has speed control by variable transformer. In one example, the ventilator 10 operates with a 90 volt dc motor. In one example, the ventilator 10 provides mechanical breath volume control by way of limiting bellows travel. In one example, the ventilator 10 includes expiratory circuit control by a cam operated valve 33. In one example, the ventilator 10 includes expiratory breath UV Disinfectant chamber 32. In one example, the ventilator 10 includes a 11.8 volt battery operated alarm 70. The alarm 70 is held in an off position by air pressure in the inspiratory circuit 20. When the circuit pressure goes to 0 or ambient pressure, an alarm sounds after a 15 second delay. In one example, the ventilator 10 and the alarm 70 are not dependent on machine electrical power. In one example, the ventilator includes the housing 50 mounted on a stand with 4 inch castors and a shelf. In one example, the ventilator 10 has a cost that is 1/10 the cost of other ventilators.

Spatially relative terms such as “under”, “below”, “lower”, “over”, “upper”, and the like, are used for ease of description to explain the positioning of one element relative to a second element. These terms are intended to encompass different orientations of the device in addition to different orientations than those depicted in the figures. Further, terms such as “first”, “second”, and the like, are also used to describe various elements, regions, sections, etc. and are also not intended to be limiting. Like terms refer to like elements throughout the description.

As used herein, the terms “having”, “containing”, “including”, “comprising” and the like are open ended terms that indicate the presence of stated elements or features, but do not preclude additional elements or features. The articles “a”, “an” and “the” are intended to include the plural as well as the singular, unless the context clearly indicates otherwise.

The present invention may be carried out in other specific ways than those herein set forth without departing from the scope and essential characteristics of the invention. The present embodiments are, therefore, to be considered in all respects as illustrative and not restrictive.

What is claimed is:

1. A ventilator comprising:
an inspiratory circuit comprising an inlet, a bellows, and an outlet port;
an expiratory circuit comprising an inlet port, and a discharge port; and
the inspiratory circuit being adjustable to control a number of breathes per time period and a volume of the breaths.

2. The ventilator of claim 1, further comprising a control module configured to control the number of breathes per time period that are output from the inspiratory circuit through the outlet port and the volume of the breaths.

3. The ventilator of claim 2, wherein the control module prevents the inspiratory circuit from moving air out of the outlet port concurrently with the expiratory circuit moving air from the patient into the inlet port.

4. The ventilator of claim 1, wherein the expiratory circuit comprises a disinfectant chamber positioned downstream from the inlet port and upstream from the discharge port, the disinfectant chamber comprising a light source that emits UV light.

5. The ventilator of claim 1, further comprising a rotating cam and a cam follower that rides along the cam and is operatively connected to the bellows to move the bellows between a contracted configuration and an expanded configuration to move the air through the inspiratory circuit.

6. The ventilator of claim 1, further comprising an arm that is adjustable to control an extent of a size of the bellows in an expanded configuration to control the volume of the breaths.

7. The ventilator of claim 1, further comprising an alarm configured to emit an alarm when the bellows falls below a predetermined size.

8. The ventilator of claim 1, further comprising a housing that extends around and protects the inspiratory and expiratory circuits, the housing comprising a front face with the outlet port of the inspiratory circuit and the inlet port of the expiratory circuit each positioned along the front face.

9. A ventilator comprising:
an inspiratory circuit comprising:
an inspiratory inlet port to receive air from the environment;
bellows positioned downstream from the inspiratory inlet port and movable between an expanded configuration and a contracted configuration; and
an outlet port positioned downstream from the bellows to deliver the air to the patient;
an expiratory circuit comprising:
an expiratory inlet port to receive the air that is exhaled from the patient; and
a discharge port to discharge the exhaled air; and
a control module comprising:
a cam to drive the bellows to adjust a number of breathes supplied to the patient; and
a lever that contacts against the bellows to control a size of the bellows in the expanded configuration to control an amount of air in each of the breaths.

10. The ventilator of claim 9, further comprising an alarm that sounds an audible signal when the bellows contracts to a size that is smaller than a predetermined amount.

11. The ventilator of claim 10, wherein the alarm comprises an arm that is pivotally mounted and contacts against the bellows.

12. The ventilator of claim 9, wherein the expiratory circuit comprises a disinfectant chamber positioned downstream from the expiratory inlet port and upstream from the discharge port, the disinfectant chamber comprising a light source that emits UV light.

13. The ventilator of claim 9, further comprising a cam follower that rides along the cam and is operatively connected to the bellows to move the bellows between the contracted configuration and the expanded configuration to move the air through the inspiratory circuit.

14. The ventilator of claim 13, further comprising an arm that is positioned adjacent to the bellows to contact against the bellows to control a size of the bellows in the expanded configuration to control the volume of the breaths.

15. The ventilator of claim 9, further comprising a housing extends around and protects the inspiratory and expiratory circuits, the housing comprising a front face with the outlet port of the inspiratory circuit and the expiratory inlet port of the expiratory circuit each positioned along the front face.

16. A method of supplying air to a patient, the method comprising:
expanding a bellows and in response drawing air into an inlet of the ventilator;
contracting the bellows and in response moving air that has been drawn into the ventilator out through an outlet port and to the patient;
receiving air that is exhaled from the patient through an inlet port;
controlling a speed at which the bellows expands and contracts and controlling a number of breaths of the air that are supplied to the patient through the outlet port; and
controlling a size of the bellows in the expanded configuration and controlling an amount of the air that is supplied to the patient in each of the breaths.

17. The method of claim 16, further comprising supplying the air to the patient and simultaneously receiving air that is exhaled from the patient through an inlet port.

18. The method of claim 16, further comprising adjusting an arm that is positioned adjacent to the bellows and controlling the size of the bellows in the expanded configuration.
